(19) United States
(12) Patent Application Publication
(10) Pub. No.: US 2017/0296738 A1
(43) Pub. Date: Oct. 19, 2017

(54) CARRIER ASSEMBLY FOR NEEDLE GUIDANCE, AND RELATED KITS FOR MOVABLY AFFIXING A SENSOR ASSEMBLY TO A BODY

(71) Applicant: Inceptio Medical Technologies, LLC, Salt Lake City, UT (US)

(72) Inventors: Bradley J. Stringer, Kaysville, UT (US); Spencer B. Shumway, South Jordan, UT (US)

(21) Appl. No.: 15/481,803

(22) Filed: Apr. 7, 2017

Related U.S. Application Data
(60) Provisional application No. 62/322,993, filed on Apr. 15, 2016.

(51) Int. Cl. A61M 5/00; A61M 5/32; A61M 5/42; A61B 17/34; A61B 90/00; A61B 90/11; G01R 33/28; A61B 5/00

(52) U.S. Cl. CPC A61M 5/00; A61M 5/427; A61M 5/3287; A61B 5/4887; A61B 90/39; A61B 90/11; G01R 33/285; A61B 17/3403

(57) ABSTRACT

A carrier assembly and related sterile kits are disclosed. The carrier assembly is configured to be used with a sensor assembly for determining a characteristic of a human body. The carrier assembly comprises a frame member comprising a sidewall defining a central opening configured to receive at least a portion of the sensor assembly and laterally extending wing members hingedly attached at a proximal end thereof to the sidewall of the frame member. The laterally extending wing members each comprise a cavity proximate a distal end thereof and configured to receive a magnet. Related kits and systems are also disclosed.

### CROSS-REFERENCE TO RELATED APPLICATION

This application claims the benefit of U.S. Provisional Patent Application Ser. No. 62/322,993, filed Apr. 15, 2016, and entitled “CARRIER ASSEMBLY FOR NEEDLE GUIDANCE, AND RELATED KITS FOR MOVABLY AFFIXING A SENSOR ASSEMBLY TO A BODY,” the disclosure of which application is hereby incorporated herein in its entirety by this reference.

### TECHNICAL FIELD

Embodiments of the disclosure relate generally to carrier assemblies and kits for placing sensor assemblies used for needle guidance, such as during cannulation of veins and arteries, and to related methods. More particularly, embodiments of the disclosure relate to systems and methods for placing a sensor assembly configured to determine one or more characteristics of a patient and to methods of movably affixing the sensor assembly to the patient.

### BACKGROUND

Insertion of needles or catheters into central veins or arteries can be a difficult task because the vein or artery may be located deep within the body or may otherwise be difficult to access in a particular patient. In addition, central veins and arteries are often in close proximity to each other, increasing a difficulty of placing the needle or catheter in the proper location without inadvertently puncturing an undesired vein or artery. Multiple attempts to access a particular vessel may result in discomfort to the patient and loss of valuable time during emergency situations.

It is known that sensors, such as ultrasonic sensors, can be used to determine the location and direction of a vessel to be penetrated or accessed. Various approaches use ultrasonic needle guidance systems to produce an image of the vessel to be penetrated using ultrasonic imaging techniques. For example, two-dimensional ultrasound imaging may be used to either mark the vessel location on the skin before attempting to access the vessel, using the known Seldinger technique, or view the vessel as the needle tip advances toward it.

Such needle guidance systems generally comprise one or more transducer assemblies (e.g., ultrasonic transducers) that are used for imaging during catheter placement or needle insertion. The guidance system, including the transducer or other imaging elements, is generally placed on the patient's skin from a sterile sheath containing a conducting medium at its tip and the sterile sheath is rolled back over the guidance system and cable. Using the needle guidance system, one or more images of a target blood vessel are obtained.

It is generally desired to secure the guidance system, including the transducer assemblies, to the patient prior to insertion of a needle and catheter placement in the target blood vessel. Undesired movement of the guidance system relative to a surface of the patient may hinder successful imaging of a desired location of the target blood vessel or successful insertion of a needle or catheter into the patient. Depending on the procedure to be performed, it may be desired to secure the guidance system to different locations of a patient's body. For example, it may be desired to place the guidance system proximate a patient's neck when internal jugular (IJ) central venous access is desired, to the patient's arm during placement of a peripherally inserted venous catheter (PICC), or proximate another location on the patient's body, such as the patient's leg. Further, the population of patients requiring cannulation or needle placement includes different body types and sizes. As only one example, a pediatric patient may have smaller features (e.g., neck, aims, legs, etc.) than an adult patient. Needle guidance systems previously known in the art generally suffer from the ability to be sufficiently secured to patients of different sizes and anatomies during such procedures.

### BRIEF SUMMARY

Embodiments disclosed herein include methods and systems of carrier assemblies to be used with needle guidance systems (such as for vascular access, biopsy, regional nerve blocks, etc.). For example, in accordance with one embodiment, a carrier assembly configured for use with a sensor assembly for determining a characteristic of a human body comprises a frame member comprising a sidewall defining a central opening configured to receive at least a portion of a sensor assembly, and laterally extending wing members each hingedly attached at a proximal end thereof to the sidewall of the frame member, the laterally extending wing members each comprising a cavity proximate a distal end thereof and configured to receive a magnet.

In additional embodiments, a kit for determining a characteristic of a human body comprises a magnetic reference element configured to be adhesively secured to the skin of a patient, and a carrier assembly configured to receive a sensor assembly. The carrier assembly comprises an integral frame member comprising a sidewall defining a central opening configured to receive at least a portion of the sensor assembly, and laterally extending wing members each hingedly attached at a proximal end thereof to the sidewall of the frame member, the laterally extending wing members each configured to be coupled to a magnet.

In yet additional embodiments, a carrier assembly for use with a needle guidance system comprises a frame member comprising a sidewall defining an internal area configured to receive at least a portion of a needle guidance system, and a pair of opposing laterally extending wing members hingedly coupled to the sidewall of the frame member, each laterally extending wing member configured to be coupled to a magnet.

### BRIEF DESCRIPTION OF THE DRAWINGS

FIG. 1A through FIG. 1C are perspective views of a sensor assembly usable with an embodiment of the disclosure;

FIG. 2A is an exploded, perspective view of a carrier assembly, in accordance with an embodiment of the disclosure;

FIG. 2B is a perspective view of the carrier assembly of FIG. 2A, in accordance with an embodiment of the disclosure;

FIG. 3A through FIG. 3C respectively comprise a top view, a side view, and a front view of a frame member of a carrier assembly, in accordance with an embodiment of the disclosure;

FIG. 4A and FIG. 4B respectively comprise a top view and a side view of a laterally extending wing members of a carrier assembly, in accordance with an embodiment of the disclosure;

FIG. 5A through FIG. 5D respectively comprise a top elevation, frontal elevation, an enlargement of a portion of the frontal elevation, and a perspective view of a magnetic reference location element suitable for use with the carrier assembly, in accordance with an embodiment of the disclosure;

FIG. GA and FIG. 6B comprise simplified schematic views of a needle guide, in accordance with an embodiment of the disclosure;

FIG. 6C is a simplified rear view of the needle guide of FIG. 6A and FIG. 6B; and

FIG. 6D is a simplified rear view of the needle guide of FIG. 6A and FIG. 6B,

### DETAILED DESCRIPTION

Illustrations presented herein are not meant to be actual views of any particular material, component, or system, but are merely idealized representations that are employed to describe embodiments of the disclosure.

The following description provides specific details, such as material types, compositions, and material thicknesses in order to provide a thorough description of embodiments of the disclosure. However, a person of ordinary skill in the art will understand that the embodiments of the disclosure may be practiced without employing these specific details. Indeed, the embodiments of the disclosure may be practiced in conjunction with conventional techniques employed in the industry. In addition, the description provided below does not form a complete sensor assembly or a complete carrier assembly configured to carry the sensor assembly or secure the sensor assembly to a patient. Further, the description provided below does not form a complete process flow for placing a needle (e.g., a biopsy needle, a regional nerve block), a central venous catheter (CVC), or both in a patient. Only those process acts and structures necessary to understand the embodiments of the disclosure are described in detail below. A person of ordinary skill in the art will understand that some components (e.g., valves, clamps, medicaments, tubing, electromagnetic radiation sources, detectors, electronic displays, and the like) are inherently disclosed herein and that adding various conventional components and acts would be in accord with the disclosure. Additional acts or materials to facilitate guidance of a needle, a catheter, or both or for securing a carrier assembly to a patient may be performed by conventional techniques.

According to embodiments disclosed herein, a carrier assembly for receiving a sensor assembly (e.g., a needle guidance system) for use during insertion of a CVC or needle such as during ultrasound-guided CVC placement, may comprise a frame member and a pair of opposing, laterally extending wing members. The frame member may include vertically extending arms that are configured to engage at least a portion of the sensor assembly when the sensor assembly is received by the carrier assembly. A needle guide may be removably secured to the frame member, for example to one of the arms, and may be configured to guide a needle at a selected angle relative to the body surface of the patient at the sensor assembly during advancement of the needle into the patient. The laterally extending wing members may be operably coupled to opposing segments of a base of the frame member. In some embodiments, the laterally extending wing members are hingedly attached to opposing segments of the base of the frame member. The laterally extending wing members may each include one or more magnets configured to cooperate with magnetically attractive elements of a reference element that may be placed on a surface of a patient's skin. The reference element may include an adhesive on one side thereof configured for attachment thereof to the skin of the patient at a location proximate a blood vessel to be cannulated. The range of movement provided by the hinged attachment of the laterally extending wing members to the frame member may facilitate securing the carrier assembly to different locations of a patient (e.g., on a neck, an arm, or a leg), or on patients having different sizes (e.g., a pediatric patient or an adult patient). Accordingly, a single carrier assembly may be sized and configured to be used for different types of patients and at different locations of a patient's body.

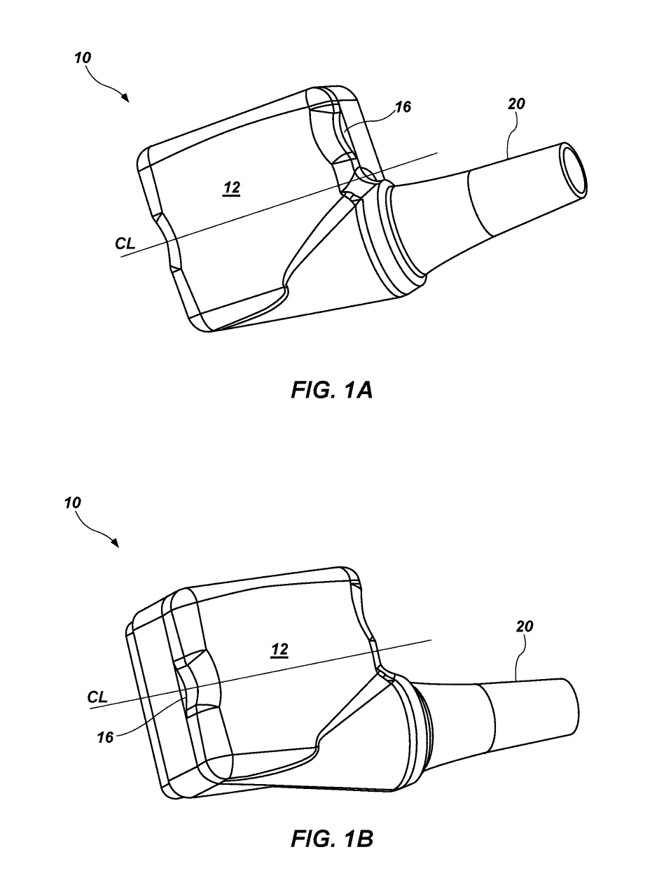

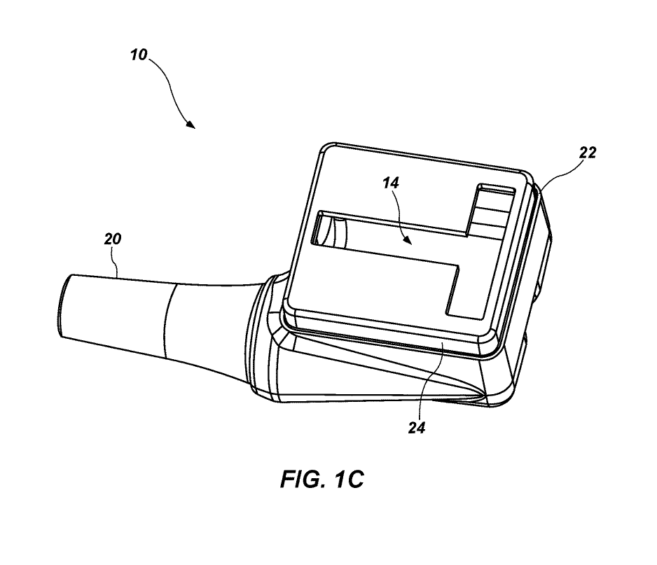

FIG. 1A through FIG. 1C are perspective views of a sensor assembly 10 that may be used determine one or more characteristics of a human body, according to embodiments of the disclosure. The sensor assembly 10 includes a housing 12 configured to house a sensor. A back side of the sensor assembly 10 may include a cut out portion 14 through which a first, linear ultrasonic elongated cross-sectional or transverse transducer array and a second linear, ultrasonic elongated longitudinal transducer array, each disposed within the housing 12, may transmit ultrasonic waves to a patient proximate a location desired to be imaged by the sensor assembly 10. The transverse transducer array and the longitudinal transducer array may each extend linearly and include a plurality of mutually adjacent piezoelectric active imaging transducer elements for transmitting and receiving ultrasonic waves, as will be understood by one having skill in the field of the present invention and similar to those described in, for example, U.S. Pat. No. 6,755,789, titled “ULTRASONIC VASCULAR IMAGING SYSTEM AND METHOD OF BLOOD VESSEL CANNULATION,” issued Jun. 29, 2004, the entire disclosure of which is incorporated herein in its entirety by this reference.

The housing 12 may include flat surfaces 16 on sides thereof, each configured to engage with an arm of a frame member to retain the housing 12 within the frame member, as will be described herein. A flat surface 16 on a first side of the housing 12 may be substantially offset from a centerline CL of the housing 12 and a flat surface 16 on an opposite, second side of the housing 12 may be centered with the centerline.

A cable cover 20 extend from a side of the housing and may be configured to carry a multi-conductor cable therein to electrically couple components of the sensor assembly 10 (e.g., the transverse transducer array and the longitudinal transducer array) to a power source.

A lower end of the housing 12 (illustrated in FIG. 1C) may include a ledge 22 extending substantially around a periphery of the housing 12 and configured to engage a surface of a frame member to retain the housing 12 therein, as will be described herein. A recessed surface 24 on sides of the housing 12 may extend substantially around an entire periphery of the housing 12 and may be configured to be surrounded by sidewalls of the frame member.

Although the sensor assembly 10 has been described above as comprising an ultrasound-guided CVC placement sensor assembly, the disclosure is not so limited and any sensor assembly may be used. The sensor assembly 10 may be configured to determine one or more characteristics of a human body, such as a location of an artery or vein, a location of one or more organs, a location of one or more bones, etc.

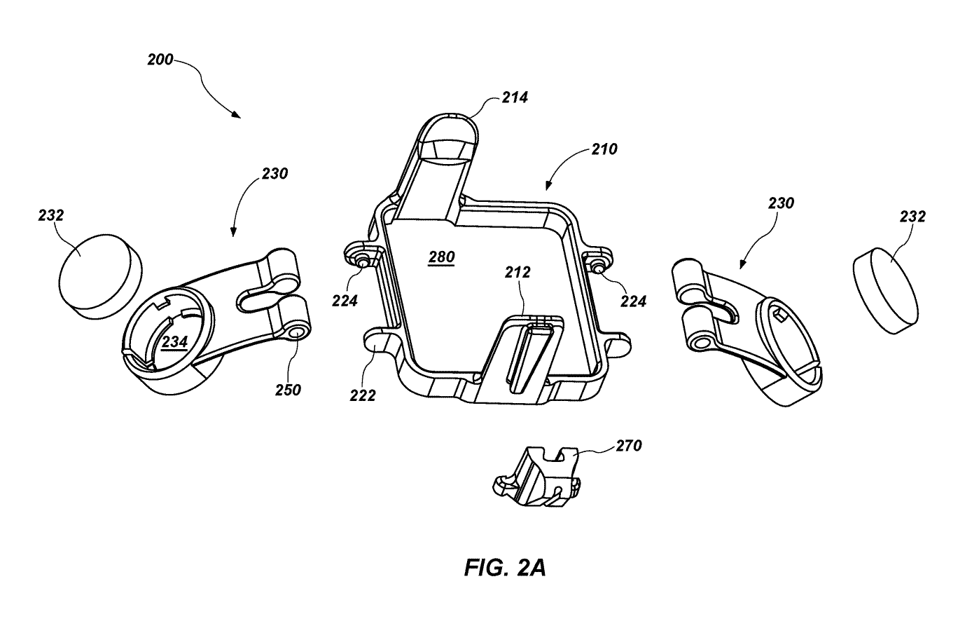

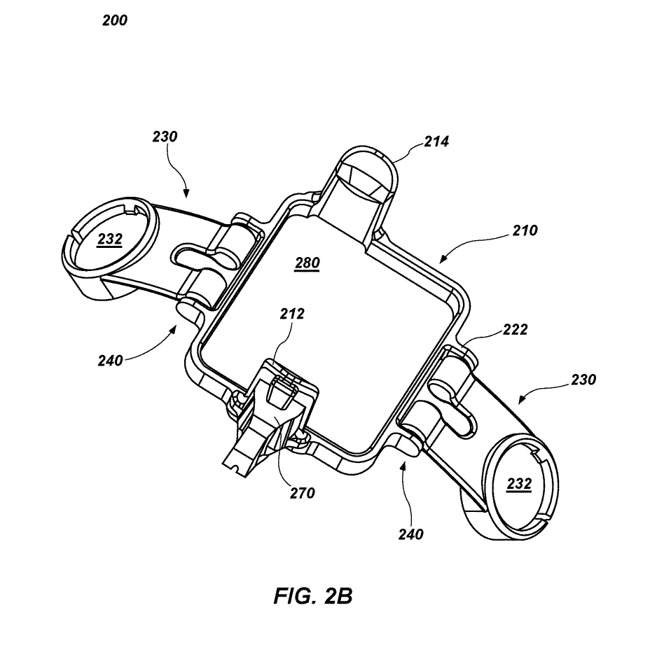

FIG. 2A is an exploded, perspective view illustrating an exploded view of a carrier assembly 200 configured to carry or house the sensor assembly 10 (FIG. 1A). The carrier assembly 200 may include a frame member 210 to which is hingedly attached a pair of laterally extending wing members 230.

The frame member 210 may be sized and shaped to receive at least a lower portion of the sensor assembly 10 (FIG. 1A) therein. The frame member 210 may include at least one surface configured to engage at least a surface of the sensor assembly 10 and retain the sensor assembly 10 within an internal area 280 defined by the base of the frame member 210. In some embodiments, the frame member 210 may include at least a first arm 212 and at least an opposing second arm 214, each protruding upwardly from the base of the frame assembly to engage the sensor assembly 10 and retain the sensor assembly 10 within the internal area 280. A needle guide 270 may be removably attached to an outer portion (i.e., a surface distal from internal area 280) of the first arm 212. The base may be sized and configured to substantially surround at least a lower portion of sides of the sensor assembly 10 when the sensor assembly 10 is disposed within the boundary of the frame member 210. In other words, the sensor assembly 10 may be placed within and contained by the frame member 210.

The laterally extending wing members 230 may be operably coupled to opposing sides of the frame member 210. In sonic embodiments, the laterally extending wing members 230 are hingedly attached to opposing sides of the frame member 210. Each laterally extending wing member 230 may be attached to a segment of the base of the frame member 210 on a side of the frame member 210 opposing the other laterally extending wing member 230. Each laterally extending wing member 230 may carry a magnet 232 disposed in a cavity 234 that is sized and shaped to receive the magnet 232 therein. The magnet 232 may be configured to interact with a magnetically active material on, for example, a magnetic reference location element that may be attached to a surface of the skin of a patient and used to secure the carrier assembly 200 to the patient, as will be described more fully herein. The frame member 210 may include hinge members 222 including mutually facing, opposing and aligned protrusions 224, each protrusion 224 sized and configured to engage an aperture 250 located proximate an end of a laterally extending wing member 230 opposite magnet 232 to form a hinge assembly 240, as illustrated in FIG. 2B.

FIG. 2B is a perspective view illustrating the carrier assembly 200 in an assembled configuration. As shown, the laterally extending wing members 230 are pivotally coupled to the frame member 210 at the hinge assemblies 240. As noted above, each hinge assembly 240 may comprise a pair of mutually aligned protrusions 224 (FIG. 2A) extending inwardly toward one another from the hinge members 222 of the frame member 210, each of which protrusions 224 is engaged with an aperture 250 of the laterally extending wing members 230. Although the hinge assemblies 240 are illustrated with the mutually aligned protrusions 224 on the frame member 210 and the laterally extending wing members 230 including the apertures 250, the disclosure is not so limited. In other embodiments, the frame member 210 may include apertures 250 configured to receive protrusions 224 of the laterally extending wing members 230 and the laterally extending wing members 230 may include protrusions 22.4 configured to be received by the apertures 250 of the frame member 210. In addition, as used herein, the term “hingedly attached” in reference to the an operable coupling between the laterally extending wing members 230 and the frame member 210 means and includes any means for providing a pivot point or a flex point of the laterally extending wing members 230 relative to the frame member 210. In some embodiments, the laterally extending wing members 230 may be sized, shaped, and configured to exhibit a greater flexibility than the frame member 210 and may be configured to conform around, for example, a patient. In some such embodiments, the carrier assembly 200 may comprise a single member comprising an integral frame member 210, wherein the laterally extending wings members 230 are integral with the frame member 210 and comprise a material having a greater flexibility than the frame member 210 or are sized and shaped to exhibit a greater flexibility than the frame member 210.

The hinge assemblies 240 may facilitate independent movement of each of the laterally extending wing members 230 about an axis of rotation relative to the frame member 210. For example, the laterally extending wing members 230 may be configured to rotate or swing about a horizontal axis parallel to an adjacent frame member segment and relative to the frame member 210 such that the magnets 232 (FIG. 2A) disposed in the laterally extending wing members 230 may engage with a magnetic reference assembly adhesively attached to the skin of a patient, regardless of whether the skin surface is substantially planar, or substantially curved, as in the case of a neck or extremity of a patient. Thus, the hinged attachment of the laterally extending wing members 230 to the frame member 210 may facilitate securement of the carrier assembly 200 to patients having different anatomies and on different portions of their bodies. Stated another way, the hinged attachment of the laterally extending wing members 230 to the frame member 210 may allow the laterally extending wing members 230 to conform to (e.g., wrap around) anatomy of a patient. The sensor assembly 10 (FIG. 1A) housed within the frame member 210 including the laterally extending wing members 230 may conform to a patient better than conventional sensor assemblies that include substantially rigid, fixed, and integral protruding members. In some embodiments, the hinged attachment of the laterally extending wing members 230 may be configured to facilitate attachment of the carrier assembly 200 to a pediatric patient as well as to a full-grown adult human being.

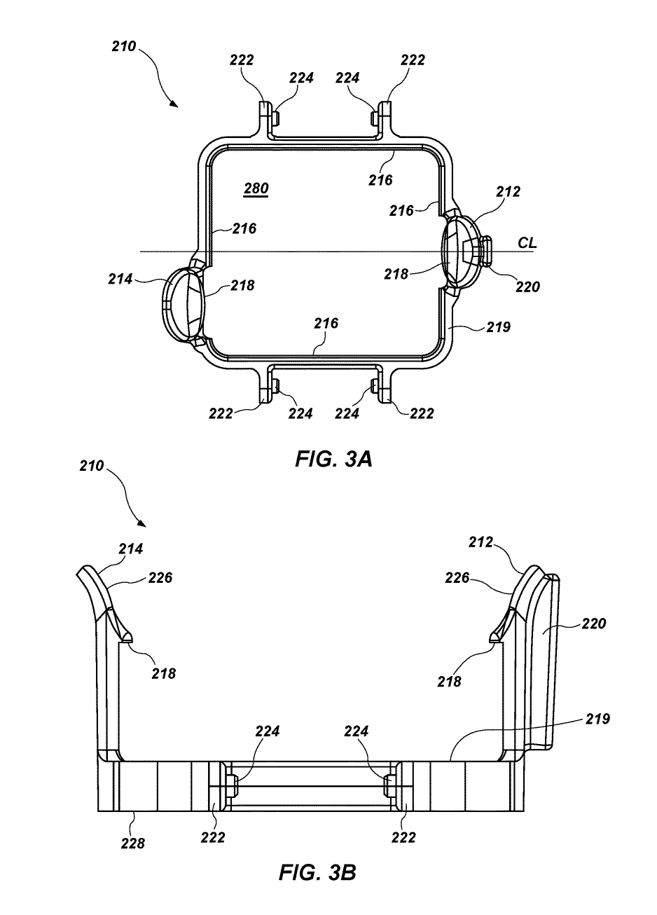

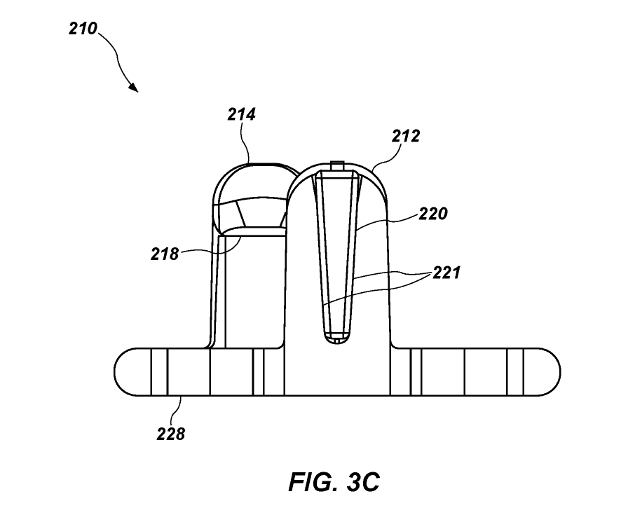

FIG. 3A is a simplified top down view of the frame member 210. The frame member 210 may include a base comprising a sidewall 216 that substantially surrounds and defines the internal area 280. The sidewall 216 may be sized and shaped to substantially surround the recessed surface 24 (FIG. 1C) of the sensor assembly. A top surface 219 of the frame member may be configured to engage the ledge 22 of the sensor assembly. The internal area 280 may be sized and shaped to receive at least a portion of the sensor assembly 10. In other words, the sidewall 216 may define the internal area 280 that substantially conforms to an outer contour of the sensor assembly 10. In some embodiments, the internal area 280 is substantially square-shaped or substantially rectangular-shaped.

The first arm 212 may extend vertically (i.e., out of the plane of :FIG. 3A) from sidewall 216 and the second arm 214 may extend substantially vertically from sidewall 216 on an opposing side of the base. In some embodiments, the first arm 212 is symmetric about a centerline CL of the frame member 210, while the second arm 214 is offset from the centerline. In some such embodiments, the frame member 210 may be asymmetric.

Each of the first arm 212 and the second arm 214 may include a protruding lip 218 (best seen in FIG. 3B) configured to extend over and substantially engage at least one portion (e.g., flat surfaces 16 (FIG. 1C)) of the sensor assembly 10. First arm 212 and second arm 214 may be formed as an integral part of frame member 210 and of a material to provide a resilient bias when a sensor assembly is inserted therein, the bias causing the first arm 212 and the second arm 214 to bend outwardly in a cantilevered manner and then return to a substantially vertical orientation with lips 218 extending over the sensor assembly 10 to securely fasten the sensor assembly 10 to the frame member 210 as well as substantially eliminate any tendency of the sensor assembly 10 to fall out of the frame member 210.

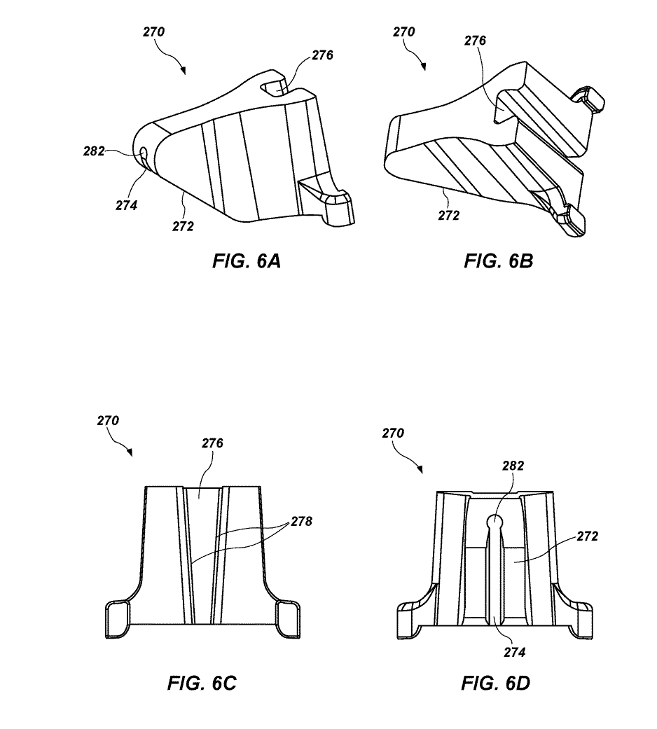

The first arm 212 may further comprise a track 220 configured to be received within an elongated groove of the needle guide 270 (FIG. 2A), as will be described with reference to FIG. 6A through FIG. 6D herein. In some embodiments, the track 220 is disposed on the first arm 212 such that the needle guide 270 is centered about the centerline CL of the frame member 210 and thus, aligned with the sensor assembly 10.

FIG. 3B is a simplified front view of the frame member 210 more clearly illustrating the protruding lip 218 on each of the first arm 212 and the second arm 214. The first arm 212 and the second arm 214 may include inwardly and downwardly angled surfaces 226 configured to guide at least a portion of the sensor assembly 10 (e.g., recessed surface 24 (FIG. 1C)) to the internal area 280 (FIG. 3A) defined by the sidewall 216. During placement of the sensor assembly 10 into the internal area 280, the first arm 212 and the second arm 214 may, as noted above, bend outwardly (i.e., away from the internal area 280) to provide space for the sensor assembly 10 within the internal area 280. When the lower portion of the sensor assembly 10 is substantially flush or coplanar with a lower surface 228 of the frame member 210, the first arm 212 and the second arm 214 may engage sides of the sensor assembly 10 and the protruding lips 218 may engage the flat surfaces 16 (FIG. 1C) to retain the sensor assembly 10 within the frame member 210.

FIG. 3C is a simplified side view of the frame member 210 illustrating the track 220 on the first arm 212. The track 220 may include opposing angled, downwardly and mutually inwardly tapering sidewalls 221 configured to be received in a groove of the needle guide 270 (FIG. 2A) so that needle guides configured, for example, for different needle sizes and for different angles of insertion may be selectably attached to the carrier assembly 200 (FIG. 2A), as will be described herein.

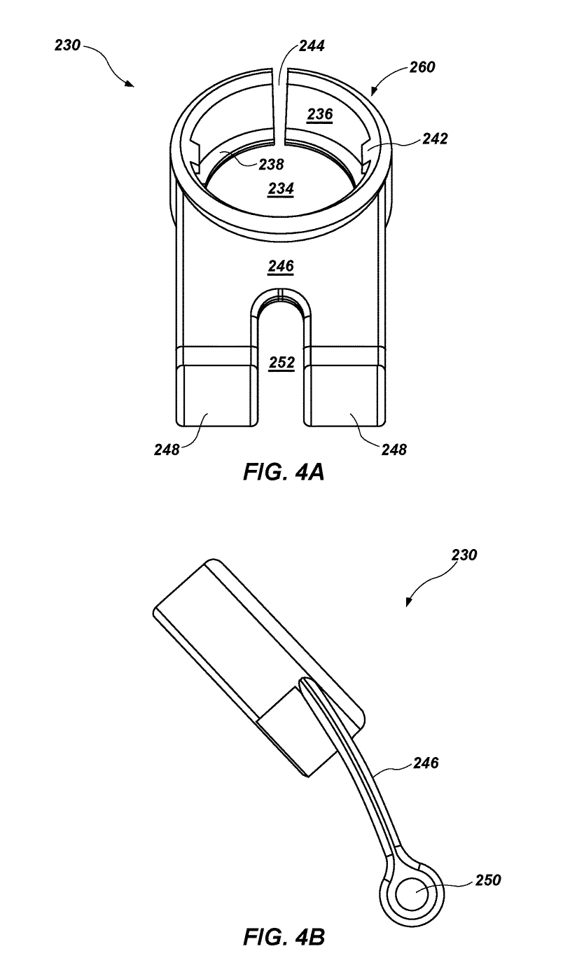

FIG. 4A is a simplified front view of one of the laterally extending wing members 230. The laterally extending wing member 230 includes at a distal end thereof, a receptacle 260 defining the cavity 234, which may be sized and shaped to receive a magnet 232 (FIG. 2A) therein. In some embodiments, the cavity 234 comprises an annular (e.g., a circular) shape. The cavity 234 may be defined by wall 236 and a lower, substantially annular, retaining ledge 238 of receptacle 260. Surfaces of the lower retaining ledge 238 may extend from the inner walls 236 toward a center portion of the cavity 232. Thus, the lower retaining ledge 238 may have a smaller diameter than a diameter defined by the inner walls 236. A magnet 232 disposed within the cavity 234 may be engaged by the walls 236 while a lower surface thereof is supported by the lower retaining ledge 238.

At the mouth of cavity 234 opposite the lower retaining ledge 238, may be located one or more protrusions 242 extending inwardly from walls 236 for securing the magnet 232 within the cavity 234. The protrusions 242 may be configured with upper surfaces downwardly and inwardly angled to guide the magnet 232 into the cavity 234 during placement of the magnet 232 in the cavity 234. The protrusions 242 may further include a surface on a lower portion thereof (i.e., a portion facing the cavity 234) located and configured to engage an upper surface of the magnet 232 when the magnet 232 is disposed within the cavity 234 during normal use and operation of the carrier assembly 200 (FIG. 2A).

In some embodiments, the receptacle 260 of the laterally extending wing member 230 may include a cutout portion 244 segmenting the wall 236 of the receptacle 260. The cutout portion 244 may facilitate insertion and removal of the magnet 232 to and from the cavity 234. In some embodiments, the wall 236 adjacent the cutout portion 244 may open (e.g., separate) resiliently as the magnet 232 is inserted into the cavity 234 and may close after the magnet 232 has been disposed within the cavity 234. Accordingly, the receptacle 260 with the cutout portion 244 may be configured to facilitate retention of the magnet 232 when the magnet is disposed in the cavity 234.

The laterally extending wing member 230 may curve arcuately away from a side of the frame member 210 to which it is pivotally secured. The laterally extending wing members 230 may include an arcuate surface 246 (best seen in FIG. 4B) configured to conform, at least partially, to a surface a patient's skin (e.g., a contour of an arm, a contour of a neck, etc.).

Although the laterally extending wing members 230 have been described and illustrated as including a receptacle 260 defining a cavity 234 sized and shaped to receive the magnet 232, the disclosure is not so limited. The magnet 232 may be attached to or coupled to the laterally extending wing members 230 by any other suitable means. By way of nonlimiting example, the magnet 232 may be adhesively attached to the laterally extending wing member 230, may be coupled to the laterally extending wing member 230 with a retention member such as a retaining clip, a retaining ring, or other suitable means for coupling the magnet 232 to the laterally extending wing member 230.

As described above, the laterally extending wing member 230 may include a plurality of apertures 250 configured to receive the protrusions 224 (FIG. 3A) of the frame member 210 (FIG. 3A) and hingedly couple the laterally extending wing member 230 to the frame member 210.

With reference again to FIG. 4A, the laterally extending wing member 230 may include a pair of legs 248 with a gap 252 therebetween. The gap 252 may be sized and configured to provide space for the legs 248 to be biased inwardly (i.e., toward each other) during attachment and removal of the laterally extending wing member 230 to the frame member 210. In some embodiments, the legs 248 may be pinched together to remove the protrusion 224 of the frame member 210 from the apertures 250 of the laterally extending wing member 230. Similarly, the legs 248 may be pinched together to create clearance for installation of the apertures 250 over the protrusions 224 and released to secure the laterally extending wing member 230 to the frame member 210.

It is contemplated that in some embodiments, the laterally extending wing members 230 may be removed and replaced with other laterally extending wing members of different lengths, curvatures, or both, configured for a particular body type or anatomical feature. It should be noted, however, that because of the hinged connection between the frame member 210 and the laterally extending wing members 230, the entire carrier assembly 200 (FIG. 2A) may substantially conform around surfaces of a variety of patients of different sizes and shapes. In other embodiments, and as described above, the laterally extending wing members 230 may be integral with the frame member 210 and may exhibit a greater flexibility than the frame member 210 such that the laterally extending wing members 230 are configured to pivot or rotate relative to the frame member 210.

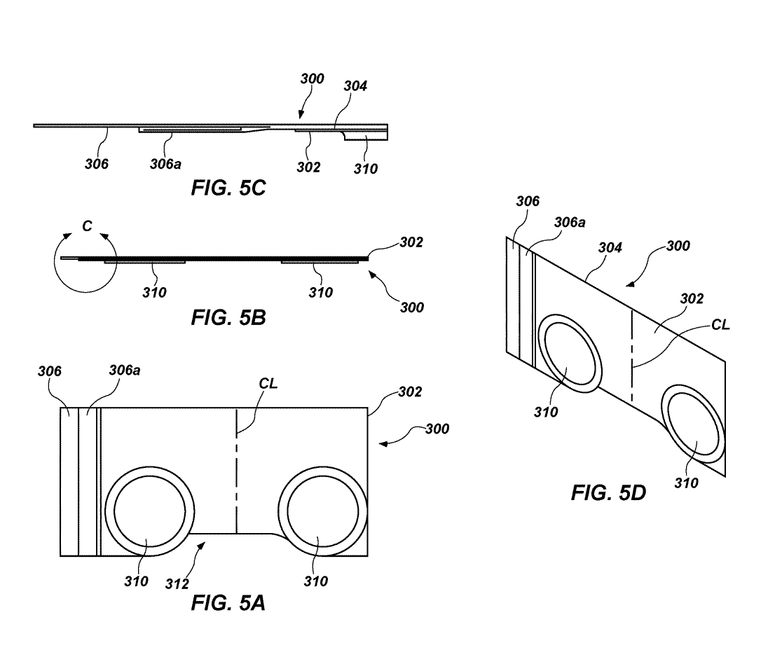

In some embodiments, the sensor assembly 10 (FIG. 1A) and carrier assembly 200 (FIG. 2B) may be used in combination with a reference location element configured for attachment to the skin of a patient over the general location of a blood vessel to be cannulated. FIG. 5A through FIG. 5D illustrate a reference location element 300 that may be used in conjunction with, for example, the sensor assembly 10 and the carrier assembly 200. The reference location element 300 may include a tape 302 having an adhesive 304 thereon. The adhesive 304 may be covered by a tape backing 306 which may include a folded portion 306a to facilitate gripping of the tape backing 306 when it is desired to remove the tape backing 306 for application of the tape 302 to the skin of a body of the patient.

The tape 302 may comprise a laminate of two individual films coated on their facing surfaces with an adhesive. Magnetically sensitive elements 310 may be disposed between opposing surfaces of the individual films. The magnetically sensitive elements 310 may be symmetrically located on each side of centerline CL of the reference location element 300. The lateral spacing of the magnetically sensitive elements 310 may, in some embodiments, approximate that of the magnets 232 (FIG. 2A) in the laterally extending wing members 230 (FIG. 2A).

The magnetically sensitive elements 310 may comprise a diameter larger than a diameter of the magnets 232 (FIG. 2A). The magnetically sensitive elements 310 may include a metal disc or flexible polymer elements, such as zinc-plated steel shim stock. In other embodiments, the magnetically sensitive elements 310 may comprise an anisotropic conductive film, such as those used in refrigerator magnets, rather than discrete magnets. The reference location element 300 may include a cutout 312 on at least one side thereof that may be sized and shaped to facilitate needle insertion into a patient without having to penetrate the tape 302.

During use and operation, the tape 302 is placed onto the skin of a patient at a location proximate a vein or artery to be cannulated. For example, the reference location element 300 may be secured to the patient by removing the tape backing 306 from the adhesive 304 and applying the tape 302 to the patient, with the adhesive-side of the tape 302 facing the patient. Magnets 232 (FIG. 2A) may he disposed on the reference location element 300 such that a magnet 232 in one laterally extending wing member 230 (FIG. 2A) is at least partially superimposed over one magnetically sensitive element 310 and a magnet 232 in the other laterally extending wing member 230 is at least partially superimposed over another magnetically sensitive element 310. Due to magnetic attraction between the magnets 232 and the magnetically sensitive elements 310, the carrier assembly 200 (FIG. 2A), including the sensor assembly 10 (FIG. 1A) may be moved laterally or vertically over the reference location element 300 to position it precisely over the patient. The magnetically sensitive elements 310 may be relatively larger than the magnets 232 (FIG. 2A) to permit the carrier assembly 200 (FIG. 213) with the sensor assembly 10 (FIG. 1A) therein to he moved about by the clinician over a limited area of the patient's body with respect to the reference location element 300 to precisely locate the sensor assembly 10 over the patient's skin. Thus, the magnets 232 may be configured to exhibit magnetic fields robust enough to maintain the sensor assembly 10 in place when it is released by the clinician.

FIG. 6A and FIG. 613 are simplified schematics of the needle guide 270 (FIG. 2A, FIG. 2B), illustrating different portions thereof. The needle guide 270 may be configured to be positioned on the first arm 212 (FIG. 2A) and may, therefore, be centered on the centerline CL (FIG. 3A) of the frame member 210 and the carrier assembly 200. The needle guide 270 may include an angled surface 272 including a guide channel 274 configured to guide a needle for insertion into a patient. The guide channel 274 may open onto angle surface 272 via slot 282 of lesser width than a diameter of guide channel 274 to enable removal of carrier assembly 200 with the sensor assembly 10 after needle placement. The needle guide 270 may further include groove 276 configured to receive a corresponding track 220 (FIG. 3C) on the first arm 212 of the frame member 210.

FIG. 6C is a rear view of the needle guide 270 illustrating the groove 276. The groove 276 may include downwardly and mutually inwardly tapering sidewalk 278 that are configured to engage tapered sides of the corresponding track 220 on the first arm 212 (FIG. 3C). In some embodiments, the downwardly and mutually inwardly tapering sidewalls 278 may be configured to engage the track 220 as the needle guide 270 is advanced along the track 220. FIG. 6D is a front view of the needle guide 270 illustrating the guide 274 configured to receive a needle.

The needle guide 270 may be removably attached to the first arm 212 (FIG. 2A) by sliding the track 220 of the first arm 212 through the groove 276 of the needle guide 270. Different needle guides 270 may be designed having different diameters of guide channels 274 and angles of incidence to a patient (when mounted to first arm 212) and therefore, needle guides having steeper or less steep angled surfaces 272 and associated guide channels 274 may be attached to the carrier assembly 200 (FIG. 2A) depending on the particular patient or the procedure being performed by the clinician,

The carrier assembly 200 (FIG. 2B) described herein may be used to facilitate securing the sensor assembly 10 (FIG. 1A) to the carrier assembly 200 and securing the carrier assembly 200 to a surface of a patient's skin. In some embodiments, the carrier assembly 200 is used in combination with the magnetic reference element 300 (FIG. 5A through FIG. 5D). The magnetic reference element 300 is adhesively secured to the skin of the patient. The carrier assembly 200 is magnetically attached to the magnetic reference element 300 with magnets 232 (FIG. 2A) disposed within laterally extending wing members 230 (FIG. 2B) of the carrier assembly 200. The laterally extending wing members 230 are hingedly attached to the frame member 210 (FIG. 2A) of the carrier assembly 200 and therefore, are configured to be secured to patients having different sizes and shapes. After an optimum ultrasound image of a vessel to be accessed is obtained through manipulation of the sensor assembly 10, the carrier assembly 200, including the sensor assembly 10, may be released, remaining secured to the patient due to magnetic attraction of magnetically sensitive elements 310 to magnets 322. A needle with a catheter attached may be inserted into the tissue at a location defined by the guide channel 274 of needle guide 270. The needle may then be inserted, guided by guide channel 274 toward a target vessel location. Accordingly, the same carrier assembly 200 may be secured to more than one type of patient and may be configured to be secured to more than one area of the body.

A kit for determining a characteristic of a human body may include the carrier assembly 200 and reference location element 300. In some embodiments, the kit comprises sterile components that may be discarded after each cannulation procedure is complete. In some embodiments, the kit includes a protective sheath into which the sensor assembly 10 may be inserted. The protective sheath may comprise an elongated tubular thin polymer film element to maintain a sterile environment, as known in the art. The sheath may be configured for disposition over the sensor assembly 10 and may comprise a length sufficient to extend over at least a portion of a cable connected to the sensor assembly. The kit may further include acoustic transmission gel. The kit may be used by a clinician to determine a location of a target vessel within a patient.

While the present disclosure has been discussed for the sake of convenience in relation to cannulation of blood vessels, it is contemplated to have equal utility in other procedures wherein a needle is inserted into a patient, such as during placement of nerve blocks, needle biopsy, or other vascular access. For example, if it is desired to block the brachial plexus (a network of nerves formed by spinal nerves C5 to C8 and T1 with contributions from C4 and T2, which constitutes the entire nerve supply for the upper extremities, as well as a number of neck and shoulder muscles), the sensor assembly of the present invention may be used to visualize the adjacent artery and vein and to avoid the artery, vein, and nerve bundle while placing the needle tip next to the nerve to initiate the block. Thus, the scope of the present disclosure encompasses the location of blood vessels for reference and locational purposes and securement of a carrier assembly to a patient, regardless of whether the blood vessels or some other structure inside the body is of interest as a target location.

While the disclosure is susceptible to various modifications and alternative forms, specific embodiments have been shown by way of example in the drawings and have been described in detail herein. However, the disclosure is not intended to be limited to the particular forms disclosed. Rather, the disclosure is to cover all modifications, equivalents, and alternatives falling within the scope of the disclosure as defined by the following appended claims and their legal equivalents.

What is claimed is:

1. A carrier assembly configured for use with a sensor assembly for determining a characteristic of a human body, the carrier assembly comprising:
a frame member comprising a sidewall defining a central opening configured to receive at least a portion of a sensor assembly; and
laterally extending wing members each hingedly attached at a proximal end thereof to the sidewall of the frame member, the laterally extending wing members each comprising a cavity proximate a distal end thereof and configured to receive a magnet.

2. The carrier assembly of claim wherein the proximal end of each laterally extending wing member comprises one of a pair of apertures or a pair of protrusions configured to operably couple to respective corresponding protrusions or apertures of the frame member to hingedly attach the laterally extending wing member to the frame member.

3. The carrier assembly of claim 2, wherein each laterally extending wing member comprises a pair of legs each having an aperture therein and a gap between the legs.

4. The carrier assembly of claim 1, wherein the sidewall of the frame member defines a square-shaped central opening or a rectangular-shaped central opening.

5. The carrier assembly of claim 1, wherein the frame member comprises at least one surface configured to engage the sensor assembly.

6. The carrier assembly of claim 5, wherein the at least one surface comprises a lip portion of one or more of a first arm and a second arm extending from the sidewall, the lip portion configured to engage a corresponding portion of the sensor assembly.

7. The carrier assembly of claim 6, wherein the laterally extending wing members are hingedly attached to the frame member at opposing segments of the sidewall that are substantially orthogonal to segments of the sidewall from which the first arm and the second arm extend.

8. The carrier assembly of claim 6, wherein the first arm extends from a sidewall opposing a sidewall from which the second arm extends.

9. The carrier assembly of claim 8, wherein the second arm is offset from a centerline of the frame member,

10. The carrier assembly of claim 8, wherein the first arm and the second arm are laterally offset from each other.

11. The carrier assembly of claim 1, further comprising a needle guide comprising an elongated groove configured to receive a track of the first arm to couple the needle guide to the first arm.

12. The carrier assembly of claim 11, wherein the nee e guide is aligned with a centerline of the frame member.

13. The carrier assembly of claim 1, wherein each laterally extending wing member comprises at least one arcuate surface between a proximal end and a distal end thereof

14. The carrier assembly of claim 1, wherein the laterally extending wing members comprise two laterally extending wing members hingedly attached to opposing sidewalls of the frame member.

15. A kit for determining a characteristic of a human body, the kit comprising:
a magnetic reference element configured to be adhesively secured to the skin of a patient; and
a carrier assembly configured to receive a sensor assembly, the carrier assembly comprising:
an integral frame member comprising a sidewall defining a central opening configured to receive at least a portion of the sensor assembly; and
laterally extending wing members each hingedly attached at a proximal end thereof to the sidewall of the frame member, the laterally extending wing members each configured to be coupled to a magnet.

16. The kit of claim 15, further comprising a protective sheath configured for disposition over the sensor assembly and of a length sufficient to extend over at least a portion of a cable connected to the sensor assembly.

17. A carrier assembly for use with a needle guidance system, the carrier assembly comprising:
a frame member comprising a sidewall defining an internal area configured to receive at least a portion of a needle guidance system; and
a pair of opposing laterally extending wing members hingedly coupled to the sidewall of the frame member, each laterally extending wing member configured to be coupled to a magnet.

18. The carrier assembly of claim 17, wherein each laterally extending wing member comprises a cavity configured to receive the magnet.

19. The carrier assembly of claim 17, wherein the frame member includes a recessed surface configured to engage the needle guidance system.

20. The carrier assembly of claim 17, wherein the pair of opposing laterally extending wing members are integral with the frame member.
